SEQUENCE LISTING (2 sequences)

SEQ ID NO: 1 - protein, 5 aa, artificial sequence
  synthetic peptide
KTTKS

SEQ ID NO: 2 - protein, 6 aa, artificial sequence
  synthetic peptide
KTTKSF

(19) United States
(12) Patent Application Publication
(10) Pub. No.: US 2018/0194805 A1
(43) Pub. Date: Jul. 12, 2018

(54) USE OF PENTAPEPTIDE-3 KTTKS OR HEXAPEPTIDE-3 KTTKSF FOR TREATING DEGENERATIVE JOINT DISEASE

(71) Applicant: Advanced Healthtech Biopeptide Laboratories Co., LTD., Taichung City (TW)

(72) Inventor: Ko-Sheng Chuang, Taichung (TW)

(21) Appl. No.: 15/405,059

(22) Filed: Jan. 12, 2017

(51) Int. Cl. C07K 7/06

(52) U.S. Cl. CPC C07K 7/06; A61K 38/00

(57) ABSTRACT

Use of pentapeptide-3 KTTKS or hexapeptide-3 KTTKSF for treating degenerative joint disease is disclosed. A pharmaceutical composition comprising an effective amount of pentapeptide-3 KTTKS or hexapeptide-3 KTTKSF and a pharmaceutically acceptable carrier is also disclosed.

### TECHNICAL FIELD OF INVENTION

The present invention relates to use of pentapeptide-3 KTTKS or hexapeptide-3 KTTKSF for treating degenerative joint disease.

### BACKGROUND

Degenerative joint disease is characterized in persistent arthropathy in articular cartilage accompanied by neoplastic hyperosteogeny. Osteophyte usually occurs on the edge of a joint or beneath articular cartilage. Development and progress of degenerative joint disease is very complicated; however, it has been found that aging among the other factors is highly associated with morbidity of degenerative joint disease. In practice, degenerative joint disease is diagnostically confirmed by a positive result from assays in wear and/or fibrosis of articular cartilage, occurrence in osteophyte and/or inflammation of soft tissues. Though it has been demonstrated that aging could induce affection in cartilage, the mechanism in the pathogenesis of degenerative joint disease has been still unclear.

Degenerative joint disease mostly occurs on joints that bear body weight, or interphalangeal joints (end knuckles in particular). Its primary clinical symptoms include pain in joints and limited movement of joints. In pathologic examination, deformation and/or swelling of joints and/or contact pressure pain in joints accompanied by snapping sound of joints are often observed.

Current medicines for treating degenerative joint disease include non-steroidal anti-inflammatory agents (such as COX2 preparations), steroids for intra-articular injection, and lubricant nutrients for intra-articular injection (i.e., hyaluronic acid preparations).

U.S. Pat. No. 4,528,133 discloses use of tetrapeptides that include two (2) or three (3) alanine (Ala; A) residues for treating arthritis.

U.S. Pat. No. 6,034,057 discloses use of cyclopeptides that include eight (8) amino acid residues and two (2) linking groups for treating rheumatoid arthritis.

U.S. Pat. No. 6,589,750 B2 and U.S. Pat. No. 7,429,448 B2 disclose use of pentapeptide QHNPR for preventing or treating water-mineral imbalance in tissues or organs (such as bones), wherein Q represents glutamine (Gln), H represents histidine (His), N represents asparagine (Asn), P represents proline (Pro), and R represents arginine (Arg).

It has not been disclosed or suggested in prior arts that a pentapeptide or hexapeptide that is rich in lysine (Lys; K) and threonine (Thr; T) is effective in treating degenerative joint disease.

Upon intense investigation, the inventor finds that pentapeptide-3 KTTKS and hexapeptide-3 KTTKSF are useful as a new therapeutic drug for treating degenerative joint disease.

The present invention therefore provides a new therapeutic approach for treating degenerative joint disease.

### SUMMARY OF INVENTION

The present invention discloses use of pentapeptide-3 KTTKS or hexapeptide-3 KTTKSF for treating degenerative joint disease.

The advantages and features of the present invention will be apparent from the following description referring to the attached drawings.

### BRIEF DESCRIPTION OF DRAWINGS

The present invention will be illustrated in detail by the preferred embodiments referring to the drawings, wherein:

FIG. 1 is a bar chart showing an effect in the DNA level of cells of cartilage of the pentapeptide KTTKS according to the preferred embodiments of the present invention;

FIG. 2 is a bar chart showing an effect in the production of collagen in cartilage tissue of the pentapeptide KTTKS according to the preferred embodiments of the present invention; and

FIG. 3 is a bar chart showing an effect in the production of proteoglycan of cartilage tissue of the pentapeptide KTTKS according to the preferred embodiments of the present invention.

### DETAILED DESCRIPTION OF THE PREFERRED EMBODIMENTS

The present invention provides use of pentapeptide-3 KTTKS or hexapeptide-3 KTTKSF for treating degenerative joint disease. The present invention further provides a pharmaceutical composition comprising an effective amount of pentapeptide-3 KTTKS or hexapeptide-3 KTTKSF and a pharmaceutically acceptable carrier. The pharmaceutically acceptable carrier may be a solid carrier, a semisolid carrier or a liquid carrier. Both of the kind and suitable amount of the solid carrier, semisolid carrier or liquid carrier have been known and conventionally used in the art. The pharmaceutical composition may be formulated in the form of tablets, pills, powder, granules, gel, paste, patches or aqueous buffers, wherein the buffers include but are not limited to phosphates and citrates.

Moreover, biochemical abbreviations are used hereinafter: “Lys; K” for lysine, “Thr; T” for threonine, “Ser; S” for serine, and “Phe; F” for phenylalanine.

Pentapeptide-3 KTTKS and hexapeptide-3 KTTKSF may be prepared by the methods disclosed in Taiwanese Patent Application Publication Nos. TW 201129368 A1 and TW 201333045 A1, for example.

As used in the present invention, all of the methods for measuring SOD, NO and MDA in synovial liquid and collagen and proteoglycan in extracellular matrix of cartilage have been known and conventionally used in the art.

### EXAMPLE 1

Animal Model

Fifteen (15) healthy adult New Zealand white rabbits were randomly divided into three (3) groups: a normal control group (Group A), a model control group (Group B) and a test group subject to intra-articular injection of a test peptide (Group C), with five (5) rabbits included in each of the groups. Fasting was conducted within four hours before surgical operations. Ketamine (10 mg/kg) and atropine (0.2 mg/kg) were injected in the muscle of each rabbit. After anesthesia took effect, each rabbit was confined to an operating table. An 8 cm-long longitudinal scission was made in the back of each knee before the skin was lifted. From the back of the knee, the articular capsule and synovial were opened. Then, the knee was bent for 90° to expose the cartilage at a lower end of the femur. Two cylindrical cutouts of 3 mm in diameter and 3 mm in depth were made. Coagulation was removed from each of the cutouts. The joint cavity was washed with a sterile saline solution. Stitches were made for each section. After the surgical operation, injection of penicillin was conducted against infection for a week. After five (5) days, each rabbit was able to eat and stand normally.

Experimental Method

Two (2) weeks after the surgical operation, the rabbits of Group A and Group B were not treated, whereas the rabbits of Group C were subject to intra-articular injection of 250 ppm/kg of pentapeptide-3 KTTKS once for every seven (7) days, and the injection lasted for twelve (12) weeks. Articular injection of 0.5 ml of saline was executed, and then 0.2 ml of synovial liquid was collected. The articular capsule was cut and opened, synovial was removed, and an entire tibial joint was taken. Arthropathy in arthrodial cartilage was observed by bare eyes and also via surgical lens. Slices were taken, dyed by hematoxylin and eosin stain (HE Stain) and toluidine blue, and then observed in an optical microscope.

Results

Results Observed by Bare Eyes

After twelve (12) weeks, in the rabbits of Group A, the surface of the knee joint was smooth and elastic, and a little synovial liquid was found in the joint cavity. In the rabbits of Group B, the injuries were partially repaired, and dents and cracks were found in the center of the injured regions. In the rabbits of Group C, the injuries were completely repaired, wherein there is almost no difference in a cross-sectional view between their knee joint and a normal knee joint, wherein the surface of their cartilage, and their cancellous bone were completely repaired, and wherein there was no sign of ossification or trace of repair.

Histomorphometric Observation

In the rabbits of Group A, the characteristics of the tissue of the knee joint were identical to those of a normal knee joint, wherein there were mature tissue of cartilage on the top, orderly arranged mature epiphyseal plates in the middle, and well-ossified cancellous bone at the bottom, and the thickness of their cartilage was normal. In the rabbits of Group B, the repaired tissues were primarily fiber-like cartilage and fibrous tissues, and there was a continuous interface between the repaired tissues and the normal tissues, wherein some of the injured regions of the knee joint were filled with a relatively large amount of fibrous tissues of cartilage, wherein there was no clear sign of epiphyseal plates, and the ossification of cancellous bone was poor. In the rabbits of Group C, the repaired tissues were not substantially different from the normal tissues of the knee joint, wherein all of the tissues of cartilage on the top, the epiphyseal plates in the middle, and the cancellous bone at the bottom were found in normal histologic morphoses, wherein the thickness of the newly born cartilage was identical to that of normal cartilage, and wherein there was no clear interface between the cartilage and the bone.

Measurement of Biochemical Indicators

As compared with the rabbits of Group B, the rabbits of Group C showed significant improvement in the morphosis of the arthrodial cartilage, wherein the SOD level in the synovial liquid was considerably increased, and the NO and MDA levels in the synovial liquid were considerably reduced.

TABLE 1
levels of NO, SOD and MDA in synovial liquid

| Group | NO (c/mol · L⁻¹) | SOD (c/mol · L⁻¹) | MDA (c/mol · L⁻¹) |
|---|---|---|---|
| A | 51.23 ± 2.55 | 6.52 ± 1.29 | 15.56 ± 3.04 |
| B | 120.55 ± 15.28 | 15.19 ± 1.48 | 24.21 ± 3.13 |
| C | 25.11 ± 3.65 | 6.18 ± 2.17 | 15.23 ± 5.62 |

### Conclusion

Regarding the histomorphometry, the injection of pentapeptide-3 KTTKS in the joint cavity considerably repaired the damaged tissues of cartilage, implying that an effect in the treatment of degenerative joint disease was clear.

Pentapeptide-3 KTTKS considerably reduced the MDA level in the synovial liquid, the lipid peroxidation injury in the joint cavity, and injuries of the cells and matrix of cartilage caused by free radicals. Hence, the arthrodial cartilage was protected.

Pentapeptide-3 KTTKS considerably increased the SOD level in the joint cavities of the test rabbits, and eliminated the free radicals that cause inflammation, thereby protecting the cartilage and suppressing and preventing cartilage affection.

Pentapeptide-3 KTTKS considerably increased the NO level in the joint cavities of the test rabbits, thereby reducing the damages in the cartilage caused by NO.

### EXAMPLE 2

Experimental Method

Firstly, cartilage cells of the growth plate of the same rabbits were taken and continuously cultivated. The cultivated cells were divided into three groups. In Group I, only 250 ppm of pentapeptide-3 KTTKS was added. In Group II, only 100 ng/ml of collagen was added. In Group III, 250 ppm of pentapeptide-3 KTTKS and 100 ng/ml of collagen were added. A negative control group was also used. After new tissues of cartilage grew at a lower portion of a centrifuge tube, the new tissues were transferred to a 24-well platefor continuing cultivation, wherein the culture medium was replaced for every 2 days. During the cultivation, samples were collected after three (3), seven (7) and fourteen (14) days, and then were analyzed.

For the histological and histochemical measurements, samples were collected, and HE dying and immunochemical measurement (S-P method) were executed.

The content of DNA in the cells of cartilage was determined by the method of Hoechst 33258.

Extracellular matrix of cartilage primarily comprises collagen and proteoglycan composed of glycosaminoglycan and core protein, in addition to water. The content of the matrix and its status of the metabolism were quantitatively analyzed by determining a level of the collagen and proteoglycan in the new tissues of cartilage.

The synthesis of the collagen of the cartilage is measured by the hydroxyproline method. Because the content of hydroxyproline in the collagen was substantially constant (about 10% by weight), an amount of hydroxyproline could be used to analyze the amount of the collagen of the cartilage by using a commercial kit including the hydroxyproline reagent. The content of the collagen in the samples was calculated.

Results

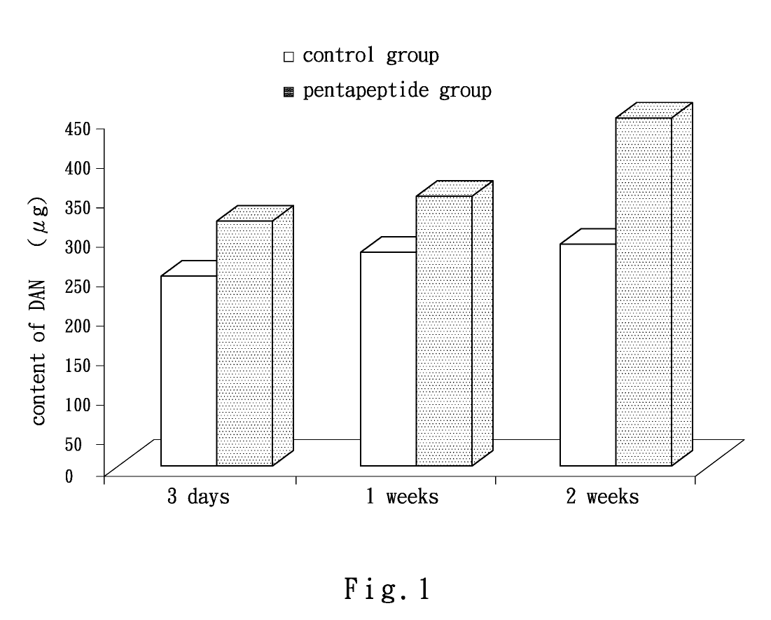

Effect on the hyperosteogeny of the cells of the cartilage of pentapeptide-3 KTTKS was determined. The pentapeptide-3 KTTKS caused considerable hyperosteogeny of the cells of the cartilage. The content of the DNA of the cells of the cartilage was shown to be statistically different from that of the control group (FIG. 1). The result showed that pentapeptide-3 KTTKS effectively improved the hyperosteogeny of the cells of the cartilage.

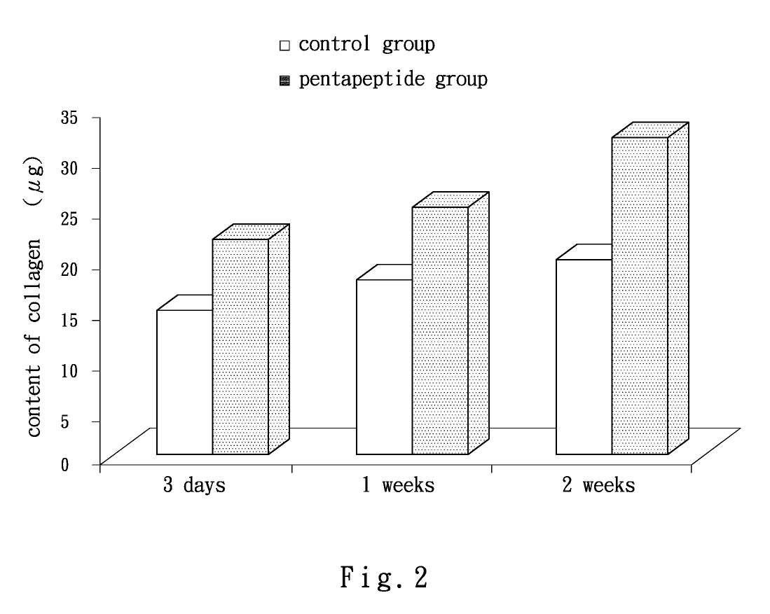

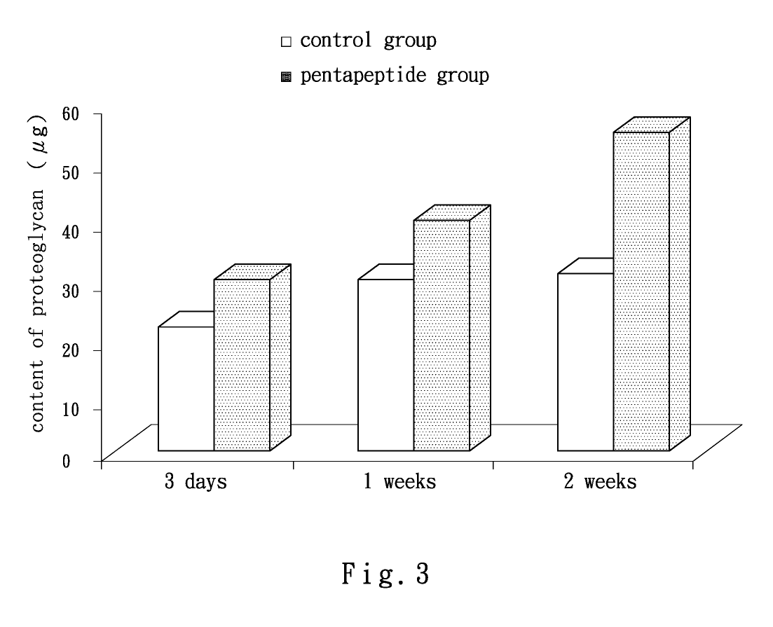

Effect on the production of the extracellular matrix of the cartilage of pentapeptide-3 KTTKS was determined. The pentapeptide-3 KTTKS considerably increased the production of the collagen and proteoglycan, and effectively improved the hyperosteogeny of the cells of the cartilage. The result statistically differs from that of the control group (FIGS. 2 and 3).

The present invention has been described via the detailed illustration of the preferred embodiments. Those skilled in the art can derive variations from the preferred embodiments without departing from the scope of the present invention. Therefore, the preferred embodiments shall not limit the scope of the present invention defined in the claims.

1. Use of pentapeptide KTTKS in the preparation of a medicament for treating degenerative joint disease.

2. The use according to claim 1, wherein after treatment, the SOD level in the synovial liquid is increased while the NO and MDA levels in the synovial liquid are reduced.

3. The use according to claim 1, wherein after treatment, the cells of the arthrodial cartilage increase.

4. A pharmaceutical composition comprising an effective amount of pentapeptide KTTKS and a pharmaceutically acceptable carrier.

5. The pharmaceutical composition according to claim 4, wherein the pharmaceutical composition is formulated in a form selected from the group consisting of tablets, pills, powder, granules, gel, paste, patches or aqueous buffer.

6. The pharmaceutical composition according to claim 5, wherein the aqueous buffer is an aqueous solution of a material selected from the group consisting of phosphate and citrate.

7. Use of hexapeptide KTTKSF in the preparation of a medicament for treating degenerative joint disease.

8. The use according to claim 7, wherein after treatment, the SOD level in the synovial liquid is increased while the NO and MDA levels in the synovial liquid are reduced.

9. The use according to claim 7, wherein after treatment, the cells of the arthrodial cartilage increase.

10. A pharmaceutical composition comprising an effective amount of hexapeptide KTTKSF and a pharmaceutically acceptable carrier.

11. The pharmaceutical composition according to claim 10, wherein the pharmaceutical composition is formulated in a form selected from the group consisting of tablets, pills, powder, granules, gel, paste, patches or aqueous buffer.

12. The pharmaceutical composition according to claim 11, wherein the aqueous buffer is an aqueous solution of a material selected from the group consisting of phosphate and citrate.
